(19) United States
(12) Patent Application Publication
(10) Pub. No.: US 2004/0077502 A1
(43) Pub. Date: Apr. 22, 2004

(54) Stable ethylene inhibiting compounds and methods for their preparation

(76) Inventors: Richard Martin Jacobson, Chalfont, PA (US); Martha Jean Kelly, Collegeville, PA (US); William Nixon James JR., Hatfield, PA (US)

Correspondence Address: Rohm and Haas Company, 100 Independence Mall West, Philadelphia, PA, 19106, US

(21) Appl. No.: 10/630,282

(22) Filed: Jul. 30, 2003

Related U.S. Application Data
(60) Provisional application No. 60/401,308, filed on Aug. 6, 2002.

(51) Int. Cl. C07C 69/74; A01N 37/00

(52) U.S. Cl. 504/313; 560/124

(57) ABSTRACT

A method to inhibit the ethylene response in plants with cyclopropene compounds by first generating stable cyclopropane precursor compounds and then converting these compounds to the gaseous cyclopropene antagonist compound by use of a reducing or nucleophilic agent.

The present invention relates to inhibiting the ethylene response in plants or plant parts. Plant parts include, for example, flowers, leaves, fruits and vegetables and may remain on the parent plant or may be harvested. The ethylene response accelerates the ripening of the plant or, especially, the harvested plant part, such as a fruit or vegetable. Such accelerated ripening makes it necessary to transport such products as quickly as possible, under optimum conditions, to the final consumer before the harvested product is rendered unmarketable by becoming prematurely rotten.

It is well known that plants contain molecular receptor sites for the molecule ethylene. Ethylene affects many plant characteristics, specifically those related to plant growth, development and senescence. For the harvester of plant products, such as fruits and vegetables, ethylene causes most problems in the area of senescence. Specifically, once fruits and vegetables are harvested, ethylene will cause these products to ripen and eventually rot at an accelerated rate. Much work has been done in an effort to either eliminate or mitigate the deleterious effects of ethylene on harvested plant products.

An example of an irreversible ethylene inhibiting agent is disclosed in U.S. Pat. No. 5,100,462. This patent discloses diazocyclopentadiene as the blocking agent. However, this compound exhibits a strong odor and is very unstable. In an effort around these problems, U.S. Pat. No. 5,518,988 discloses the discovery of cyclopropene and derivatives thereof, which are used as effective blocking agents for the ethylene binding site. However, while the compounds of this patent do not suffer from the odor problems of diazocyclopentadiene they are relatively unstable gases. Therefore, the stability of these gases, as well as the explosive potential these gases pose when compressed still present problems.

Since the cyclopropenes of the '988 patent have proven to be very effective ethylene inhibitors, it remains very desirable to find a viable means to resolve their instability problem. One approach that was taken is disclosed in U.S. Pat. No. 6,017,849. This patent shows that it is possible to encapsulate the cyclopropene molecule into a cyclodextrin molecule as a carrier. This approach allows for the safe storage and transport of the cyclopropene/cyclodextrin complex, in general providing a shelf life of more than one year.

Although the foregoing encapsulation technique provides a substantially more stable ethylene inhibiting agent, problems still remain. For instance, the double bond in the cyclopropene molecule is very reactive and makes the molecule susceptible to degradation under a variety of storage and handling conditions.

Therefore, what is needed is an ethylene inhibitor that is storage stable over a long period of time, is not susceptible to self-degradation and eliminates the significant risk of explosion associated with the handling of cyclopropenes. The present invention solves these problems by utilizing certain precursors of the cyclopropene class of ethylene inhibitor molecules. These precursors have increased storage stability. In practice, the precursors are converted to their corresponding cyclopropene molecule when treatment of the target plant parts is desired.

The present invention comprises a method of stabilizing unstable cyclopropene molecules by converting them to their more stable cyclopropane analogs. The double bond is eliminated by binding moieties to each carbon atom component of the double bond. In the formulae of the disclosure of this invention, these moieties are designated as W1 and W2. These stabilizing moieties are selected from F, Cl, Br, I, alkoxy, acyloxy, alkoxycarbonyloxy, aminocarbonyloxy, alkylaminocarbonyloxy, dialkylaminocarbonyloxy, alkylsulfonyloxy and arylsulfonyloxy groups; with the proviso that at least one of W1 and W2 is a Br or I.

Specifically, the present invention comprises a method of generating cyclopropene derivatives of structures I, II, III and IV for use as plant ethylene response inhibitors. These compounds are represented as follows:

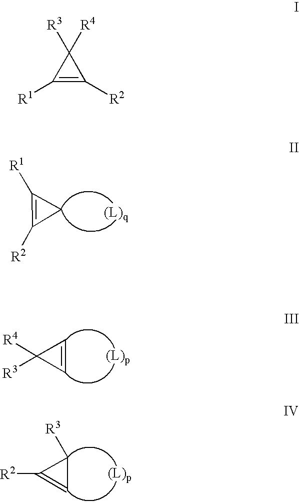

Structures I, II, III and IV represent cyclopropene derivative compounds which are effective ethylene antagonists. These compounds can be derived from their respective cyclopropane precursor molecules V, VI, VII and VIII:

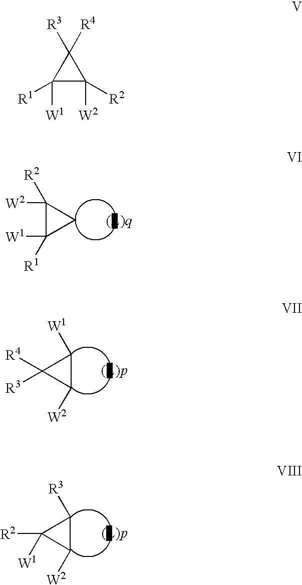

Compounds of structures V, VI, VII and VIII are reacted with a reducing agent or a nucleophile to obtain the respective gaseous compounds of structures I, II, III, and IV. Compounds I, II, III and IV are thus released into the target enclosed atmosphere to treat the plants or plant parts to inhibit the ethylene response.

The present invention comprises the cyclopropane compounds of structures V, VI, VII and VIII wherein:

a) each R¹, R², R³, and R⁴ is independently a group of the formula:

—(L)ₙ—Z

wherein:

i) p is an integer from 3 to 10;

q is an integer from 4 to 11;

n is an integer from 0 to 12;

ii) each L is independently selected from a member of the group D, E, or J wherein:

D is of the formula:

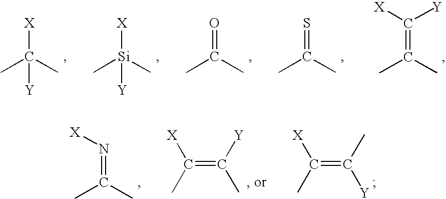

E is of the formula:

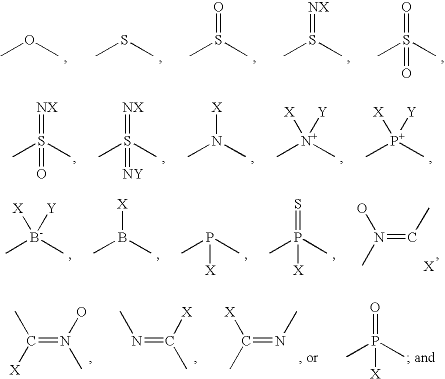

J is of the formula:

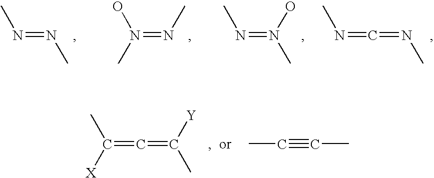

wherein:

A) each X and Y is independently a group of the formula:

—(L)ₘ—Z

and

B) m is an integer from 0 to 8; and

C) no more than two E groups are adjacent to each other and no J groups are adjacent to each other;

iii) each Z is independently selected from:

A) hydrogen, halo, cyano, nitro, nitroso, azido, chlorate, bromate, iodate, isocyanato, isocyanido, isothiocyanato, pentafluorothio, or

B) a group G, wherein G is an unsubstituted or substituted; unsaturated, partially saturated, or saturated; monocyclic, bicyclic, tricyclic, or fused; carbocyclic or heterocyclic ring system wherein;

1) when the ring system contains a 3 or 4 membered heterocyclic ring, the heterocyclic ring contains 1 heteroatom;

2) when the ring system contains a 5, or more, membered heterocyclic ring or a polycyclic heterocyclic ring, the heterocyclic or polycyclic heterocyclic ring contains from 1 to 4 heteroatoms;

3) each heteroatom is independently selected from N, O, and S;

4) the number of substituents is from 0 to 5 and each substituent is independently selected from X;

b) W¹ and W² are selected from F, Cl, Br, I, alkoxy, acyloxy, alkoxycarbonyloxy, aminocarbonyloxy, alkylaminocarbonyloxy, dialkylaminocarbonyloxy, alkylsulfonyloxy, and arylsulfonyloxy;

c) at least one of W¹ and W² is a Br or I; and

d) the total number of non-hydrogen atoms in each compound is 50 or less;

its enantiomers, stereoisomers, salts, and mixtures thereof; or a composition thereof.

For the purposes of this invention, in the structural representations of the various L groups, each open bond indicates a bond to another L group, a Z group, or the cyclopropene moiety. For example, the structural representation

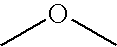

indicates an oxygen atom with bonds to two other atoms; it does not represent a dimethyl ether moiety.

Typical R¹, R², R³, and R⁴ groups include, for example: alkenyl, alkyl, alkynyl, acetylaminoalkenyl, acetylaminoalkyl, acetylaminoalkynyl, alkenoxy, alkoxy, alkynoxy, alkoxyalkoxyalkyl, alkoxyalkenyl, alkoxyalkyl, alkoxyalkynyl, alkoxycarbonylalkenyl, alkoxycarbonylalkyl, alkoxycarbonylalkynyl, alkylcarbonyl, alkylcarbonyloxyalkyl, alkyl(alkoxyimino)alkyl, carboxyalkenyl, carboxyalkyl, carboxyalkynyl, dialkylamino, haloalkoxyalkenyl, haloalkoxyalkyl, haloalkoxyalkynyl, haloalkenyl, haloalkyl, haloalkynyl, hydroxyalkenyl, hydroxyalkyl, hydroxyalkynyl, trialkylsilylalkenyl, trialkylsilylalkyl, trialkylsilylalkynyl, dialkylphosphonato, dialkylphosphato, dialkylthiophosphato, dialkylaminoalkyl, alkylsulfonylalkyl, alkylthioalkenyl, alkylthioalkyl, alkylthioalkynyl, dialkylaminosulfonyl, haloalkylthioalkenyl, haloalkylthioalkyl, haloalkylthioalkynyl, alkoxycarbonyloxy; cycloalkenyl, cycloalkyl, cycloalkynyl, acetylarninocycloalkenyl, acetylaminocycloalkyl, acetylaminocycloalkynyl, cycloalkenoxy, cycloalkoxy, cycloalkynoxy, alkoxyalkoxycycloalkyl, alkoxycycloalkenyl, alkoxycycloalkyl, alkoxycycloalkynyl, alkoxycarbonylcycloalkenyl, alkoxycarbonylcycloalkyl, alkoxycarbonylcycloalkynyl, cycloalkylcarbonyl, alkylcarbonyloxycycloalkyl, carboxycycloalkenyl, carboxycycloalkyl, carboxycycloalkynyl, dicycloalkylamino, halocycloalkoxycycloalkenyl, halocycloalkoxycycloalkyl, halocycloalkoxycycloalkynyl, halocycloalkenyl, halocycloalkyl, halocycloalkynyl, hydroxycycloalkenyl, hydroxycycloalkyl, hydroxycycloalkynyl, trialkylsilylcycloalkenyl, trialkylsilylcycloalkyl, trialkylsilylcycloalkynyl, dialkylaminocycloalkyl, alkylsulfonylcycloalkyl, cycloalkylcarbonyloxyalkyl, cycloalkylsulfonylalkyl, alkylthiocycloalkenyl, alkylthiocycloalkyl, alkylthiocycloalkynyl, dicycloalkylaminosulfonyl, haloalkylthiocycloalkenyl, haloalkylthiocycloalkyl, haloalkylthiocycloalkynyl; aryl, alkenylaryl, alkylaryl, alkynylaryl, acetylaminoaryl, aryloxy, alkoxyalkoxyaryl, alkoxyaryl, alkoxycarbonylaryl, arylcarbonyl, alkylcarbonyloxyaryl, carboxyaryl, diarylamino, haloalkoxyaryl, haloaryl, hydroxyaryl, trialkylsilylaryl, dialkylaminoaryl, alkylsulfonylaryl, arylsulfonylalkyl, alkylthioaryl, arylthioalkyl, diarylaminosulfonyl, haloalkylthioaryl; heteroaryl, alkenylheteroaryl, alkylheteroaryl, alkynylheteroaryl, acetylaminoheteroaryl, heteroaryloxy, alkoxyalkoxyheteroaryl, alkoxyheteroaryl, alkoxycarbonylheteroaryl, heteroarylcarbonyl, alkylcarbonyloxyheteroaryl, carboxyheteroaryl, diheteroarylamino, haloalkoxyheteroaryl, haloheteroaryl, hydroxyheteroaryl, trialkylsilylheteroaryl, dialkylaminoheteroaryl, alkylsulfonylheteroaryl, heteroarylsulfonylalkyl, alkylthioheteroaryl, heteroarylthioalkyl, diheteroarylaminosulfonyl, haloalkylthioheteroaryl; heterocyclyl, alkenylheteroycycyl, alkylheteroycycyl, alkynylheteroycycyl, acetylaminoheterocyclyl, heterocyclyloxy, alkoxyalkoxyheterocyclo, alkoxyheterocyclyl, alkoxycarbonylheterocyclyl, heterocyclylcarbonyl, alkylcarbonyloxyheterocyclyl, carboxyheterocyclyl, diheterocyclylamino, haloalkoxyheterocyclyl, haloheterocyclyl, hydroxyheterocyclyl, trialkylsilylheterocyclyl, dialkylaminoheterocyclyl, alkylsulfonylheterocyclyl, alkylthioheterocyclyl, heterocyclylthioalkyl, diheterocyclylaminosulfonyl, haloalkyllthioheterocyclyl; hydrogen, fluoro, chloro, bromo, iodo, cyano, nitro, nitroso, azido, chlorato, bromato, iodato, isocyanato, isocyanido, isothiocyanato, pentafluorothio; acetoxy, carboethoxy, cyanato, nitrato, nitrito, perchlorato, allenyl; butylmercapto, diethylphosphonato, dimethylphenylsilyl, isoquinolyl, mercapto, naphthyl, phenoxy, phenyl, piperidino, pyridyl, quinolyl, triethylsilyl, trimethylsilyl; and substituted analogs thereof.

Typical G groups include, for example: saturated or unsaturated cycloalkyl, bicyclic, tricyclic, polycyclic, saturated or unsaturated heterocyclic, unsubstituted or substituted phenyl, naphthyl, or heteroaryl ring systems such as, for example, cyclopropyl, cyclobutyl, cyclopent-3-en-1-yl, 3-methoxycyclohexan-1-yl, phenyl, 4-chlorophenyl, 4-fluorophenyl, 4-bromophenyl, 3-nitrophenyl, 2-methoxyphenyl, 2-methylphenyl, 3-methyphenyl, 4-methylphenyl, 4-ethylphenyl, 2-methyl-3-methoxyphenyl, 2,4-dibromophenyl, 3,5-difluorophenyl, 3,5-dimethylphenyl, 2,4,6-trichlorophenyl, 4-methoxyphenyl, naphthyl, 2-chloronaphthyl, 2,4-dimethoxyphenyl, 4-(trifluoromethyl)phenyl, 2-iodo-4-methylphenyl, pyridin-2-yl, pyridin-3-yl, pyridin-4-yl, pyrazinyl, pyrimidin-2-yl, pyrimidin-4-yl, pyrimidin-5-yl, pyridazinyl, triazol-1-yl, imidazol-1-yl, thiophen-2-yl, thiophen-3-yl, furan-2-yl, furan-3-yl, pyrrolyl, oxazolyl, isoxazolyl, thiazolyl, isothiazolyl, oxadiazolyl, thiadiazolyl, quinolyl, isoquinolyl, tetrahydrofuryl, pyrrolidinyl, piperidinyl, tetrahydropyranyl, morpholinyl, piperazinyl, dioxolanyl, dioxanyl, indolinyl and 5-methyl-6-chromanyl, adamantyl, norbornyl, and their substituted analogs such as, for example: 3-butyl-pyridin-2-yl, 4-bromo-pyridin-2-yl, 5-carboethoxy-pyridin-2-yl, 6-methoxyethoxy-pyridin-2-yl.

Preferably, two of R¹, R², R³, and R⁴ are hydrogen. More preferably, R¹ and R² are hydrogen or R³ and R⁴ are hydrogen. Even more preferably, R², R³, and R⁴ are hydrogen or R¹, R², and R³ are hydrogen. Most preferably, R², R³, and R are hydrogen.

Preferably, n is from 0 to 8. Most preferably, n is from 1 to 7. Preferably, m is 0 to 4. Most preferably, m is from 0 to 2.

Preferably, D is —CXY—, —SiXY—, —CO—, or —CS—. More preferably D is —CXY—. Preferably, E is —O—, —S—, —NX—, or —SO₂—. Preferably, X and Y are independently H, halo, OH, SH, —C(O)(C₁-C₄)alkyl-, —C(O)O(C₁-C₄)alkyl-, —O—(C₁-C₄)alkyl, —S—(C₁-C₄)alkyl, or substituted or unsubstituted (C₁-C₄)alkyl. Preferably, Z is H, halo, or G. More preferably, Z is H or G.

Preferably, each G is independently a substituted or unsubstituted; five, six, or seven membered; aryl, heteroaryl, heterocyclic, or cycloalkyl ring. More preferably, each G is independently a substituted or unsubstituted phenyl, pyridyl, cyclohexyl, cyclopentyl, cycloheptyl, pyrolyl, furyl, thiophenyl, triazolyl, pyrazolyl, 1,3-dioxolanyl, or morpholinyl. Even more preferably, G is unsubstituted or substituted phenyl, cyclopentyl, cycloheptyl, or cyclohexyl. Most preferably, G is cyclopentyl, cycloheptyl, cyclohexyl, phenyl, or substituted phenyl wherein the substituents are independently selected from 1 to 3 of methyl, methoxy, and halo.

The method of the present invention comprises converting the precursor compounds of structures V, VI, VII and VIII into the corresponding ethylene antagonistic compounds of structures I, II, III, and IV, respectively. This is achieved by reacting the compound of structures V, VI, VII or VIII with a reducing or a nucleophilic agent. The moieties identified as W1 and W2 on structures V, VI, VII and VIII are often referred to as “leaving groups”. These groups will remain on the core molecule until cleaved off by reaction with, as in this instance, a reducing or nucleophilic agent. Once the reducing or nucleophilic agent cleaves off the leaving group, the molecule of structures V, VI, VII and VIII converts to the molecule of structures I, II, III and IV, respectively.

Reducing agents may be classified as metals, organometallic reagents and low valent metal ions. Suitable examples of metals are zinc, magnesium, iron, copper, samarium and aluminum. Examples of organometallic reagents are methyllithium and n-butyllithium. Low valent metal ions include Cr(II), Ti(II), Cu(I) and Fe(II). The most preferred reducing agent is metallic zinc.

Nucleophilic agents include mercaptans, selenides, phosphines, phosphites, Na2S, Na2Te, Na2S2O4, diethylphosphite sodium salt, KSCN, NaSeCN, thiourea, diphenyltelurium and NaI. These nucleophiles may also be incorporated into polymeric reagents.

Molecules of structure V are preferred in the practice of this invention. The most preferred molecule is where R1=CH3, R2=H, R3=H, R4=H, W1=I and W2=I. This molecule is identified as 1,2-diiodo-1-methylcyclopropane. In the practice of this invention, this molecule represents a stable precursor to the ethylene antagonist 1-methylcylopropene. The following reaction shows the conversion from the stable 1,2-diiodo-1-methylcyclopropane to the gaseous 1-methylcylopropene upon reaction with zinc.

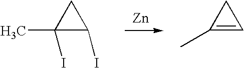

A number of examples were prepared. Different leaving groups are also exemplified. Although 77 examples were actually prepared, it is only necessary to show a few reaction schemes. The number of the example correlates with the same number in the list of structures identified.

### EXAMPLE 23

### 1,1,2-tribromocyclopropane

Into a 3000 ml three necked round bottomed flask equipped with a mechanical stirrer was added 350 g of bromoform, 575 g of methylene chloride, 130 g of vinyl bromide, 4.5 g of N,N′-dibenzyl-N,N,N′,N′-tetramethylethylenediammonium dibromide and 60 g of 45% aqueous potassium hydroxide. After stirring for two days, 500 ml of water was added and the organic layer was separated. An additional 4.5 g of N,N′-dibenzyl-N,N,N′,N′-tetramethylethylenediammonium dibromide and 60 g of 45% aqueous potassium hydroxide were added and stirring was resumed overnight. After washing with water, the organic layer was distilled yielding 1,1,2-tribromocyclopropane bp (10 torr) 75-80° C. nmr (CDCl₃) δ 1.72 (t, 1H), 2.76 (t, 1H), 3.58 (t, 1H).

### EXAMPLE 37

### Preparation of 1-Hexyl-1,2,2-tribromocyclopropene

a. 2-Bromo-oct-1-ene

A solution of 9.42 ml (0.0728 mol) of 2,3-dibromopropene in 70 ml diethylether was placed under a nitrogen atmosphere by use of a Firestone valve. While cooling in an ice water bath, a solution of 0.091 mol of pentylmagnesium bromide in 70 ml diethyl ether was added slowly via addition funnel. After stirring for 2 hours while warming to room temperature, there was then added via syringe 50 ml of 1 N hydrochloric acid to the reaction cooling in an ice water bath. The resulting mixture was transferred to a separatory funnel and the phases were separated. The organic layer was dried over MgSO₄ and filtered. The solvent was removed from the filtrate in vacuo to yield 15.0 g (85.7% of theory) of 81% pure 2-bromo-oct-1-ene as an oil.

b. 1,1,2-Tribromo-2-hexyl-cyclopropane

To 5.42 g (28.4 mmol) of 2-bromo-oct-1-ene in 7.42 ml (85.1 mmol) of bromoform and 48.8 ml of methylene chloride, were added 1.30 g (2.84 mmol) of N,N′-dibenzyl-N,N,N′,N′-tetramethylethylenediammonium dibromide and 12.1 ml (142 mmol) of 45% aqueous potassium hydroxide. The mixture was stirred at room temperature for 5 days. There was then added hexanes and water. This mixture was filtered. The resulting mixture was transferred to a separatory funnel and the phases were separated. The organic layer was dried over MgSO₄ and filtered. The solvent was removed from the filtrate in vacuo to yield 5.25 g (51.0% of theoretical) of 1,1,2-tribromo-2-hexyl-cyclopropane as an oil.

### EXAMPLE 55

### 1,2-Diiodo-1-octylcyclopropane

To 20 g of methyl alcohol was added 1.33 g (16.2 mmole) of anhydrous sodium acetate and 3.3 g (13 mmole) of elemental iodine. The mixture was cooled to 5° C. whereon 2.0 g of 1-octylcyclopropene (13 mmole) [prepared from 1,2,2-tribromo-1-octylcyclopropane by the method of Baird, Mark S.; Hussain, Helmi H.; Nethercott, William; J.Chem.Soc.Perkin Trans. 1, 1986, 1845-1854] The reaction was stirred at room temperature for two hours. The reaction was concentrated in vacuo and the product was diluted with hexanes and washed with dilute aqueous sodium hydroxide. Re-concentration in vacuo and column chromatography over silica gel gave 1.7 g of the desired 1,2-diiodo-1-octylcyclopropane. nmr (CDCl₃) δ 0.88 (m, 4H), 1.3 (m, 10H), 1.5-1.8 (m, 5H), 3.26 (t, 1H).

### EXAMPLE 56

### 1,2-Diiodo-1-benzylcyclopropane

1-benzylcyclopropene [prepared from 3.65 g (10.0 mmole) of 1,2,2-tribromo-1-benzylcyclopropane by the method of Baird, Mark S.; Hussain, Helmi H.; Nethercott, William; J.Chem.Soc.Perkin Trans. 1, 1986, 1845-1854] was added to a stirred mixture of 0.77 g (9.4 mmole) of anhydrous sodium acetate and 2.60 g of elemental iodine in 30 g of methanol. After stirring overnight, the reaction was concentrated in vacuo and the product was diluted with hexanes and washed with dilute aqueous sodium hydroxide. Re-concentration in vacuo and column chromatography over silica gel gave 3.0 g of the desired 1,2-diiodo-1-benzylcyclopropane. nmr (CDCl₃)δ 1.18 (t, 1H), 3.1 (abq, 2H), 3.41 (t, 1H), 7.3 (m, 5H).

### EXAMPLE 60

### 1,2-Diiodo-1-methylcyclopropane

To 300 g of methyl alcohol was added 8.2 g (100 mmole) of anhydrous sodium acetate and 53 g (209 mmole) of elemental iodine. The mixture was cooled to 5° C. whereon 19 g of 1-methylcyclopropene [prepared from 3-chloro-2-methyl-propene; see, for example, Hopf, H.; Wachholz, G.; Walsh, R. Chem. Ber., 118, 3579 (1985), and Köster, R et al., Liebigs Annalen Chem., 1219-1235, (1973).] was added. The reaction was stirred at room temperature until the color lightened. The reaction was concentrated in vacuo and the product was diluted with hexanes and washed with dilute aqueous sodium hydroxide. Re-concentration in vacuo gave 45.7 g of the desired 1,2-diiodo-1-methylcyclopropane. Bp (5 torr) 76° C. nmr (CDCl₃)δ 0.88 (t, 1H), 1.71 (t, 1H), 1.99 (s, 3H), 3.22 (t, 1H).

### EXAMPLE 61

### 1,1-Dichloro-2-bromocyclopropane

Into a 3000 ml three necked round bottomed flask equipped with a mechanical stirrer was added 500 g of chloroform, 103 g of vinyl bromide, 5.6 g of N,N′-dibenzyl-N,N,N′,N′-tetramethylethylenediammonium dibromide and 200 g of 45% aqueous potassium hydroxide. After stirring for two days, 500 ml of water was added and the organic layer was separated. The organic layer was distilled yielding 1,1-dichloro-2-bromocyclopropane bp (760 torr) 140-150° C. nmr (CDCl₃) δ 1.65 (t, 1H), 2.13 (t, 1H), 3.53 (t, 1H).

### EXAMPLE 76

### 1,2-Diiodocyclopropane

Cyclopropane, made from 10 ml of allyl chloride by the method of Binger [J. Org. Chem. 61, 6462-6464 (1996)] was condensed into a flask containing 10.13 g of iodine, 2 g of pyridine and 100 g of 2-propanol at −70° C. The reaction mixture was slowly warmed to +10° C. over the course of three hours and concentrated in vacuo. The resulting mixture was partitioned between diethyl ether and dilute aqueous hydrochloric acid. Washing the ether layer with dilute aqueous sodium hydroxide, saturated aqueous sodium chloride, drying over anhydrous magnesium sulfate, and concentration in vacuo yielded 6.0 g of trans-1,2-diiodocyclopropane which was purified by column chromatography over silica gel. nmr (CDCl₃) δ 1.36 (t, 2H), 2.66 (t, 2H).

Structural examples of compounds produced according to the invention.

| | | | | |
|---|---|---|---|---|
| W1 | W2 | R2 | R3 | R4 |

| | |
|---|---|
| | Structure class V |
| 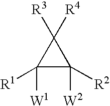 | |

| | | | | | | |
|---|---|---|---|---|---|---|
| | R1 | | | | | |
| 1 | OCTYL | Br | Br | Br | H | H |
| 2 | C6H5 | Br | Br | Br | H | H |
| 3 | CH2CH2C6H5 | Br | Br | Br | H | H |
| 4 | OCTYL | Br | Cl | CL | H | H |
| 5 | CH2OC6H5 | Br | Br | Br | H | H |
| 6 | C8H17 | Br | Cl | Cl | H | H |
| 7 | CH2OC6H4OMe-4 | Br | Br | Br | H | H |
| 8 | CH2C6H5 | Br | Br | Br | H | H |
| 9 | UNDECYL | Br | Br | Br | H | H |
| 10 | NONYL | Br | Br | Br | H | H |
| 11 | HEPTYL | Br | Br | Br | H | H |
| 12 | DECYL | Br | Br | Br | H | H |
| 13 | (2-CYCLOHEXYLETHYL) | Br | Br | Br | H | H |
| 14 | TRIDECYL | Br | Br | Br | H | H |
| 15 | (3-ETHYLHEPTYL) | Br | Br | Br | H | H |
| 16 | (CYCLOHEPTYLMeTHYL) | Br | Br | Br | H | H |
| 17 | (CYCLOHEXYLMeTHYL) | Br | Br | Br | H | H |
| 18 | CH2OC6H4CL-4 | Br | Br | Br | H | H |
| 19 | CH2CH2OH | Br | Br | Br | H | H |
| 20 | CH2OCH2CH2OCH2CH2OMe | Br | Br | Br | H | H |
| 21 | CH2CH2CO2ET | Br | Br | Br | H | H |
| 22 | Br | Br | OET | H | H | H |
| 23 | Br | Br | Br | H | H | H |
| 24 | Br | Br | OBU | Br | H | H |
| 25 | CH2C6H4Me-4 | Br | Br | Br | H | H |
| 26 | CH2CH2CH2C6H5 | Br | Br | Br | H | H |
| 27 | CH2C6H4OMe-2 | Br | Br | Br | H | H |
| 28 | HEPTYL(7-OMe) | Br | Br | Br | H | H |
| 29 | HEPTYL(6-Me) | Br | Br | Br | H | H |
| 30 | CH2CH20PENTYL | Br | Br | Br | H | H |
| 31 | HEPTYL(7-OH) | Br | Br | Br | H | H |
| 32 | CH2CH2CH2CH2C6H5 | Br | Br | Br | H | H |
| 33 | PENTYL | Br | Br | Br | H | H |
| 34 | CH2THIOPHENE-2-YL | Br | Br | Br | H | H |
| 35 | BUTYL | Br | Br | Br | H | H |
| 36 | CH2CH2C6H4CL-4 | Br | Br | Br | H | H |
| 37 | HEXYL | Br | Br | Br | H | H |
| 38 | CH2C6H4Me-3 | Br | Br | Br | H | H |
| 39 | HEPTYL(4,6,6-TRIMETHYL) | Br | Br | Br | H | H |
| 40 | HEXYL(6-CO2H) | Br | Br | Br | H | H |
| 41 | CH2CYCLOPENTYL | Br | Br | Br | H | H |
| 42 | HEXYL(6-OMS) | Br | Br | Br | H | H |
| 43 | Br | Br | Br | H | OCTYL | H |
| 44 | PENTADECYL | Br | Br | Br | H | H |
| 45 | (CH2)4CF3 | Br | Br | Br | H | H |
| 46 | CH2CH2CO2H | Br | Br | Br | H | H |
| 47 | NONYL(4,8-Me2) | Br | Br | Br | H | H |
| 48 | DODECYL | Br | Br | Br | H | H |
| 49 | CH2CH2COMORPHOLINE | Br | Br | Br | H | H |
| 50 | CH2CH(ET)BU | Br | Br | Br | H | H |
| 51 | (CH2)7CN | Br | Br | Br | H | H |
| 52 | (CH2)7NET2 | Br | Br | Br | H | H |
| 53 | TETRADECYL | Br | Br | Br | H | H |
| 54 | TETRADECYL | Br | Br | Br | H | H |
| 55 | OCTYL | I | I | H | H | H |
| 56 | BENZYL | I | I | H | H | H |
| 57 | (3,3-DIMETHYLBUTYL) | Br | Br | Br | H | H |
| 58 | HEXYL | Br | Br | Br | HEXYL | H |
| 59 | METHYL | Br | Br | Br | H | H |
| 60 | METHYL | I | I | H | H | H |
| 61 | Cl | Cl | Br | H | H | H |
| 62 | CH2CH2CH2DIOXANE-2-YL | Br | Br | Br | H | H |
| 63 | CH2CH2CONET2 | Br | Br | Br | H | H |
| 64 | CH2SIET3 | Br | Br | Br | H | H |
| 65 | CH2CH2OCH(Me)OET | Br | Br | Br | H | H |
| 66 | CH2CH2OSO2PH | Br | Br | Br | H | H |
| 67 | (CH2)6SiMe3 | Br | Br | Br | H | H |
| 68 | (CH2)2SiMe3 | Br | Br | Br | H | H |
| 69 | CH2CH2CO2CH2OAC | Br | Br | Br | H | H |
| 70 | C(Me)(Me)C6H5 | Cl | Br | Br | H | H |
| 71 | (CH2)6SiMe2Ph | Br | Br | Br | H | H |
| 72 | CH2Ph | Br | Cl | Cl | H | H |
| 73 | Me | Br | Br | Me | Me | Me |
| 74 | (CH2)4OCOC6H4Me-4 | Br | Br | Br | H | H |
| 75 | (CH2)4OH | Br | Br | Br | H | H |
| 76 | H | I | I | H | H | H |

| | | | | Structure Class VIII | |
|---|---|---|---|---|---|
| 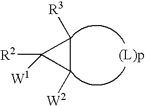 | | | | | |
| | (L)ₚ | | | | |
| 77 | Ch2Ch2CH2CH2CH2 | Br | Br | Br | H |

### Chemically Induced Release of a Cyclopropene

### Control Experiment

Into a 50 ml Florence flask with magnetic stirring was placed 2 ml of tetrahydrofuran and 0.30 g of 1,2-diiodo-1-methylcyclopropane. After stirring for 5 minutes GC analysis of the headspace showed no detectable 1-methylcyclopropene. GC method uses Varian CP-PoraBOND Q column 10 meters long 0.32 mm ID; helium carrier; initial temperature 50° C.; initial time 0 minutes; ramp rate 20° C./min; final temperature 270° C.; final time 5 minutes; injection volume 0.20 ml. The retention time of an authentic sample of 1-methylcyclopropene was 2.91 minutes. 1 ppm is easily detectable under these conditions.

### 1-Methylcyclopropene Formation Using Zinc Metal in Tetrahydrofuran

Into a 100 ml Florence flask with magnetic stirring was placed 2 ml of tetrahydrofuran and 1.0 g of zinc dust. The zinc was activated with 10 drops of 1,2-dibromoethane. Then 0.34 g of 1,2-diiodo-1-methylcyclopropane was added. After stirring for 20 hours, GC analysis of the headspace showed 4658 ppm 1-methylcyclopropene.

### 1-Methylcyclopropene Formation Using Zinc Metal in Methanol

Into a 100 ml Florence flask with magnetic stirring was placed 2 ml of methanol and 1.0 g of zinc dust. The zinc was activated with 10 drops of 1,2-dibromoethane. Then 0.34 g of 1,2-diiodo-1-methylcyclopropane was added. After stirring for 30 minutes GC analysis of the headspace showed 98390 ppm 1-methylcyclopropene.

### 1-Methylcyclopropene Formation Using Magnesium Metal

Into a 100 ml Florence flask with magnetic stirring was placed 2 ml of tetrahydrofuran and 1.1 g of magnesium turnings. The magnesium was activated with 10 drops of 1,2-dibromoethane. Then 0.35 g of 1,2-diiodo-1-methylcyclopropane was added. After stirring for 3 hours GC analysis of the headspace showed 49993 ppm 1-methylcyclopropene.

### 1-Methylcyclopropene Formation Using Triphenylphosphine

Into a 50 ml Florence flask with magnetic stirring was placed 3 g of dimethylformamide and 1.2 g of triphenylphosphine. Then 0.83 g of 1,2-diiodo-1-methylcyclopropane was added. After stirring for 15 minutes at room temperature, GC analysis of the headspace showed 10 ppm 1-methylcyclopropene.

### 1-Methylcyclopropene Formation Using 4-Methylbenzenethiol

Into a 100 ml Florence flask with magnetic stirring was placed 2 g of dimethylformamide, 0.70 g of potassium t-butoxide, and 0.84 g of 4-methylbenzenethiol. Then 0.40 g of 1,2-diiodo-1-methylcyclopropane was added. After stirring for 15 minutes at room temperature, GC analysis of the headspace showed 87567 ppm 1-methylcyclopropene.

### 1-Methylcyclopropene Formation Using Polymer Containing Benzenethiol Groups

The polymeric reagent was prepared by slurrying 50 ml of Duolite™ GT73 (Rohm and Haas Company) and stirring for two hours with 50 ml of water and 10 g of 45% aqueous potassium hydroxide. The slurry was filtered, washed twice with water, thrice with methanol, air dried, and placed in a vacuum oven overnight. 0.54 g of this polymeric reagent was placed in a 122 ml vial and the beads were wetted with 0.10 g of 1,2-diiodo-1-methylcyclopropane in 0.70 g of methanol. After standing overnight at room temperature, GC analysis of the headspace showed 134 ppm of 1-methylcyclopropene.

We claim:

1. A method of stabilizing cyclopropene compounds by converting them to their cyclopropane analogs comprising covalently bonding to each carbon atom component of the double bond in the cyclopropene compound a moiety W1 and W2, respectively, wherein W1 and W2 are each selected from the group consisting of F, Cl, Br, I, alkoxy, acyloxy, alkoxycarbonyloxy, aminocarbonyloxy, alkylaminocarbonyloxy, dialkylaminocarbonyloxy, alkylsulfonyloxy and arylsulfonyloxy, with the proviso that at least one of W1 and W2 is Br or I.

2. A cyclopropane compound comprising a structure selected from the group consisting of: 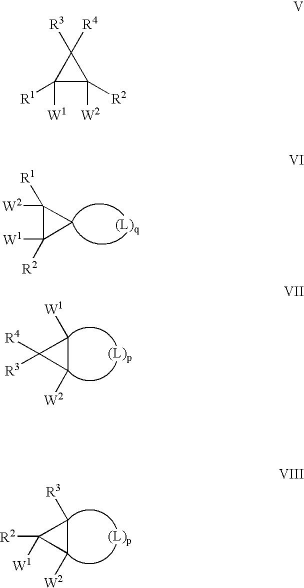
wherein:
a) each R¹, R², R³, and R⁴ is independently a group of the formula:
—(L)ₙ—Z
i) p is an integer from 3 to 10;
q is an integer from 4 to 11;
n is an integer from 0 to 12;
ii) each L is independently selected from a member of the group D, E, or J D is of the formula: 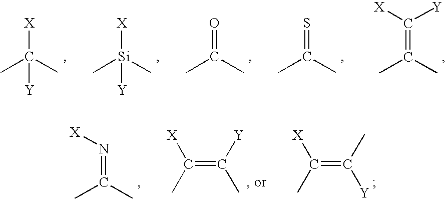
E is of the formula: 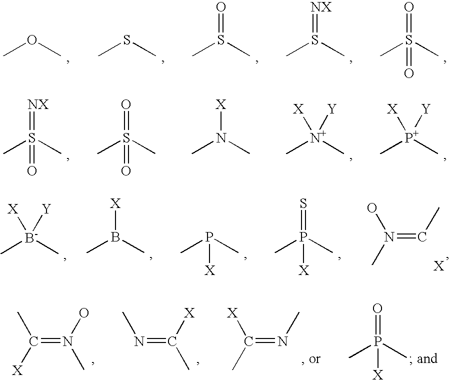
J is of the formula: 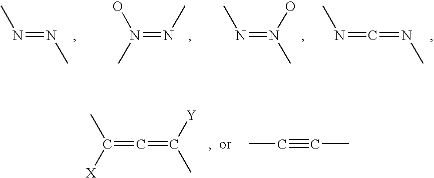
A) each X and Y is independently a group of the formula:
—(L)ₘ—Z;
and
B) m is an integer from 0 to 8; and
C) no more than two E groups are adjacent to each other and no J groups are adjacent to each other;
iii) each Z is independently selected from:
A) hydrogen, halo, cyano, nitro, nitroso, azido, chlorate, bromate, iodate, isocyanato, isocyanido, isothiocyanato, pentafluorothio, or
B) a group G, wherein G is an unsubstituted or substituted; unsaturated, partially saturated, or saturated; monocyclic, bicyclic, tricyclic, or fused; carbocyclic or heterocyclic ring system wherein;
1) when the ring system contains a 3 or 4 membered heterocyclic ring, the heterocyclic ring contains 1 heteroatom;
2) when the ring system contains a 5, or more, membered heterocyclic ring or a polycyclic heterocyclic ring, the heterocyclic or polycyclic heterocyclic ring contains from 1 to 4 heteroatoms;
3) each heteroatom is independently selected from N, O, and S;
4) the number of substituents is from 0 to 5 and each substituent is independently selected from X;
b) W¹ and W² are selected from F, Cl, Br, I, alkoxy, acyloxy, alkoxycarbonyloxy, aminocarbonyloxy, alkylaminocarbonyloxy, dialkylaminocarbonyloxy, alkylsulfonyloxy, and arylsulfonyloxy;
c) provided that at least one of W¹ and W² is I; and
d) the total number of non-hydrogen atoms is 50 or less.

3. The compound of claim 2 wherein each of W1 and W2 are I.

4. The compound 1,2-diiodo-1-methylcyclopropane.

5. A process to generate a compound of structure I, II, III or IV 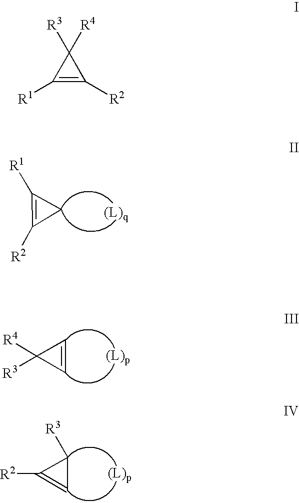
comprising contacting a compound of structure V, VI, VII or VIII of claim 2, with a reducing or nucleophilic agent to convert the compound of structure V, VI, VII or VIII into its respective analogous compound of structure I, II, III or IV, respectively, wherein:
a) each R¹, R², R³, and R⁴ is independently a group of the formula:
—(L)ₙ—Z
i) p is an integer from 3 to 10;
q is an integer from 4 to 11;
n is an integer from 0 to 12;
ii) each L is independently selected from a member of the group D, E, or J:
D is of the formula: 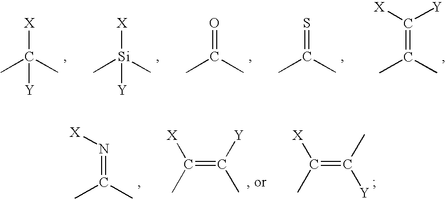
E is of the formula: 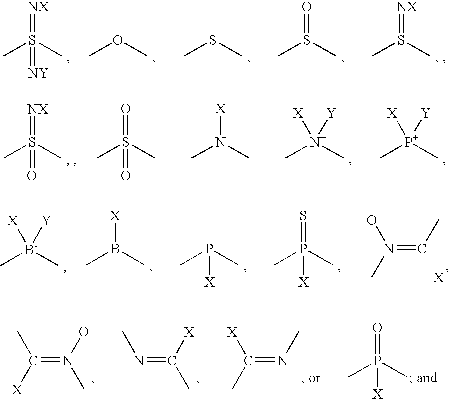
J is of the formula: 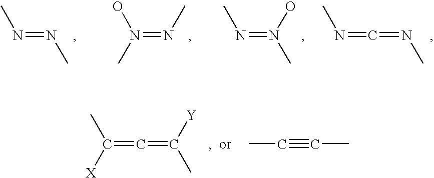
A) each X and Y is independently a group of the formula:
—(L)ₘ—Z;
and
B) m is an integer from 0 to 8; and
C) no more than two E groups are adjacent to each other and no J groups are adjacent to each other;
iii) each Z is independently selected from:
A) hydrogen, halo, cyano, nitro, nitroso, azido, chlorate, bromate, iodate, isocyanato, isocyanido, isothiocyanato, pentafluorothio, or
B) a group G, wherein G is an unsubstituted or substituted; unsaturated, partially saturated, or saturated; monocyclic, bicyclic, tricyclic, or fused; carbocyclic or heterocyclic ring system wherein;
1) when the ring system contains a 3 or 4 membered heterocycli ring, the heterocyclic ring contains 1 heteroatom;
2) W¹ and W² are selected from F, Cl, Br, I, alkoxy, acyloxy, alkoxycarbonyloxy, aminocarbonyloxy, alkylaminocarbonyloxy, dialkylaminocarbonyloxy, alkylsulfonyloxy, and arylsulfonyloxy;
c) provided that at least one of W¹ and W² is a Br or I; and
d) the total number of non-hydrogen atoms is 50 or less.

6. The method of claim 5 wherein the reducing agent is selected from the group consisting of metals, organometallic reagents and low valent metal ions.

7. The method of claim 5 wherein the nucleophilic agent is selected from the group consisting of mercaptans, selenides, phosphines, phosphites, Na2S, Na2Te, Na2S2O4, diethylphosphite sodium salt, KSCN, NaSeCN, thiourea, diphenyltelurium and NaI.

8. A method of using any one of a compound of structure V, VI VII and VIII of claim 2 as a plant ethylene response antagonist by contacting the plant with said compound.
